(19) United States
(12) Patent Application Publication
(10) Pub. No.: US 2007/0243211 A1
(43) Pub. Date: Oct. 18, 2007

(54) Compositions for regulating metabolic disorders and methods of use thereof

(76) Inventor: Russell M. Jaffe, Sterling, VA (US)

Correspondence Address: COOLEY GODWARD KRONISH LLP;ATTN: Patent Group, Suite 500, 1200 - 19th Street, NW, WASHINGTON, DC, 20036-2402, US

(21) Appl. No.: 11/593,639

(22) Filed: Nov. 7, 2006

Related U.S. Application Data
(60) Provisional application No. 60/733,780, filed on Nov. 7, 2005.

(51) Int. Cl. A61K 36/03; A61P 3/00; C08B 37/04

(52) U.S. Cl. 424/195.17; 536/3

(57) ABSTRACT

The invention relates to compositions comprising a combination of ingredients including one or more oxidative fat metabolizers, neurotransmitters, algin or algin equivalents and medium chain triglycerides that are useful in regulating disorders and maintaining healthy metabolism. The compositions of the invention are useful in enhancing metabolism, burning fat, and enhancing energy.

### RELATED APPLICATIONS

This application claims the benefit of U.S. Provisional Application 60/733,780, filed Nov. 7, 2005, which is herein incorporated by reference in its entirety for all purposes.

### FIELD OF THE INVENTION

The invention relates to compositions comprising a combination of ingredients including one or more oxidative fat metabolizers, one or more neurotransmitters, one or more algin or algin equivalents, and one or more medium chain triglycerides that are useful in regulating disorders and maintaining healthy metabolism. The compositions of the invention are useful in enhancing metabolism, burning fat, and increasing energy.

### BACKGROUND OF THE INVENTION

A majority of diseases that are a major concern for public health involve faulty glucose metabolism. One of the primary molecules that mediate glucose metabolism is insulin. A hormone excreted from the pancreas, insulin, loses its effectiveness in stimulating cells to absorb glucose from the blood. Once this happens, glucose levels remain elevated for extended periods of time after food is consumed. The pancreas will continue to secrete insulin for an extended period in an attempt to compensate for the elevated glucose levels.

An increase in glucose levels in the liver can lead to posttranslational activation of several key enzymes of glycolysis and lipogenesis, including fructose-6-phosphate 2-kinase/fructose-2,6-bisphosphatase, fatty acid synthase, acetyl-CoA carboxylase, and L-type pyruvate kinase (LPK). A high-carbohydrate diet also induces transcription of many of the genes encoding these enzymes, thereby promoting long-term storage of sugars as triglycerides and an increased risk of weight gain or obesity (Goodridge, Annu. Rev. Nutr. 7:157-185 (1987) and Granner & Pilkis, J. Biol. Chem. 265:10173-10176 (1990)).

Obesity, hyperlipidemia, and diabetes have been shown to play a causal role in various disorders including, for example, atherosclerotic cardiovascular diseases, which currently account for a considerable proportion of morbidity in Western society.

One human disorder, termed “Syndrome X” or “Metabolic Syndrome,” is manifested by defective glucose metabolism (e.g., insulin resistance), elevated blood pressure (i.e., hypertension), and a blood lipid imbalance (i.e., dyslipidemia). See e.g. Reaven, 1993, Annu. Rev. Med. 44:121-131.

There is a clear need to develop safer natural therapies that are efficacious at lowering serum cholesterol, increasing HDL serum levels, preventing coronary heart disease, and/or treating existing disease such as atherosclerosis, obesity, diabetes, and other diseases that are affected by glucose metabolism and/or elevated glucose levels.

### SUMMARY OF THE INVENTION

The invention encompasses compositions that are useful in regulating disorders related to metabolism.

In one embodiment, the invention encompasses compositions comprising one or more oxidative fat metabolizers, one or more neurotransmitters, one or more algins or algin equivalents, and one or more medium chain triglycerides (“MCT”).

In another embodiment, the invention encompasses a kit for regulating a condition in a mammal comprising a container comprising at least the following components: one or more oxidative fat metabolizers; one or more neurotransmitters; one or more algins or algin equivalents; one or more medium chain triglycerides; and instructions for use, wherein each of the components is pre-measured into a respective unit of use amount.

A method of treating metabolism related disorders comprising administering to a subject in need thereof an effective amount of a composition comprising one or more oxidative fat metabolizers, one or more neurotransmitters, one or more algins or algin equivalents, and one or more medium chain triglycerides.

### DETAILED DESCRIPTION OF THE INVENTION

Definitions

As used herein and unless otherwise indicated, the phrase “regulating metabolism” indicates an observable (i.e., measurable) change in at least one aspect of metabolism including, but not limited to, total blood lipid content, blood HDL cholesterol, blood LDL cholesterol, blood VLDL cholesterol, blood triglyceride, blood Lp(a), blood apo A-I, blood apo E or blood non-esterified fatty acids.

As used herein and unless otherwise indicated, the phrase “altering metabolism” indicates an observable (i.e., measurable) change in at least one aspect of metabolism including, but not limited to, total blood glucose content, blood insulin, the blood insulin to blood glucose ratio, insulin sensitivity, or oxygen consumption.

As used herein and unless otherwise indicated, the phrase “effective amount” of a composition of the invention is measured by the effectiveness of a compound of the invention, wherein at least one adverse effect of a disorder or condition is ameliorated or alleviated.

As used herein the terms diluent, adjuvant, excipient, filler or carrier includes any additional additive or combinations of additives to the compositions of the present invention. Non-limiting examples of diluents, adjuvants, excipients, fillers or carriers can be liquids, such as water and oils, including those of petroleum, animal, vegetable or synthetic origin, such as peanut oil, soybean oil, mineral oil, sesame oil and the like, gum acacia, gelatin, starch paste, talc, keratin, colloidal silica, urea, and the like, stabilizing, thickening, lubricating and coloring agents, flavoring agents, etc., saline solutions and aqueous dextrose and glycerol solutions, various types of starch, various types of sugars such as glucose, lactose, and sucrose, malt, rice, flour, chalk, silica gel, sodium stearate, glycerol monostearate, sodium chloride, calcium carbonate, calcium phosphate, dried skim milk, glycerol, propyleneglycol, polyethylene glycol, ethanol and the like. The present compositions, if desired, can also contain minor amounts of wetting or emulsifying agents, or pH buffering agents.

As used herein, “preventative measure,” “preventing” or “prevention” refers to a reduction of the risk of acquiring a given disorder. The compositions of the present invention are also suitable for preventing conditions or disorders, as described herein.

### Compositions of the Invention

The invention encompasses compositions that are useful in regulating, altering, treating, and preventing various disorders, particularly disorders of the metabolism as described herein. In one embodiment, the compositions of the present invention comprise one or more oxidative fat metabolizers, one or more neurotransmitters, one or more algins or algin equivalents, and one or more MCTs.

In another embodiment, the compositions of the present invention comprise an oxidative fat metabolizer, a neurotransmitter, an algin or algin equivalent, a MCT, and optionally phosphatidylcholine, inositol, ethanolamine, turmeric, beeswax, gelatin, and vegetable glycerin, glycerol ethyl ester and water. The term “glycerol ethyl ester” refers to the condensation product of glycerol and ethanol.

In another embodiment of the compositions of the present invention, the oxidative fat metabolizer is carnitine; carnitine includes, but is not limited to, L-carnitine; the neurotransmitter is gamma amino butyric acid (“GABA”); the algin or algin equivalent is kelp extract. Optionally, the composition can further contain an excipient or filler, although, the compositions can be used without an excipient or filler.

In another embodiment, the compositions of the present invention comprise an oxidative fat metabolizer, a neurotransmitter, an algin or algin equivalent, a MCT, and optionally phosphatidylcholine, inositol, ethanolamine, turmeric, beeswax, gelatin, vegetable glycerine, glycerol ethyl ester, water and combinations thereof which may include any or all of the optional ingredients (e.g., exipients, fillers, etc, as described herein.

In one embodiment of the invention, the compositions of the present invention comprise L-carnitine, GABA, kelp extract, and MCT, for example one or more MCTs from coconut oil.

In another embodiment, the compositions of the present invention comprise L-carnitine, GABA, kelp extract, MCT from coconut oil, phosphatidylcholine, turmeric, beeswax, gelatin for example kosher gelatin, and glycerine, for example vegetable glycerine, from palm fruit.

In another embodiment, the compositions of the present invention comprise of L-carnitine, GABA, kelp extract, MCT from coconut oil, phosphatidylcholine, turmeric, beeswax, gelatin, and vegetable glycerine from palm fruit.

In another embodiment, the compositions of the present invention comprise L-carnitine, GABA, kelp extract, MCT from coconut oil, phosphatidylcholine, turmeric, beeswax, gelatin, and vegetable glycerine from palm fruit and excipients or fillers as described herein.

In another embodiment, the compositions of the present invention comprise: an oxidative fat metabolizer; a neurotransmitter; an algin or algin equivalent; and a medium chain triglyceride.

In another embodiment, the compositions of the present invention comprise: an oxidative fat metabolizer; a neurotransmitter; an algin or algin equivalent; a medium chain triglyceride; and optionally at least one of the following: phosphatidylcholine, inositol and ethanolamine; turmeric; beeswax; gelatin; glycerol; glycerol ethyl ester; excipients or fillers as described herein; and combinations thereof which may include any or all of the optional ingredients.

In another embodiment, the compositions of the present invention comprise: an oxidative fat metabolizer; a neurotransmitter; an algin or algin equivalent; a medium chain triglyceride; phosphatidylcholine, inositol and ethanolamine; turmeric; beeswax; gelatin; glycerol; glycerol ethyl ester; and excipients or fillers as described herein.

In another embodiment, the compositions of the present invention comprise: an oxidative fat metabolizer which is L-carnitine; a neurotransmitter which is gamma amino butyric acid; an algin or algin equivalent from kelp extract; a medium chain triglyceride from coconut oil; phosphatidylcholine, inositol and ethanolamine; turmeric; beeswax; gelatin; glycerol from palm fruit; one or more glycerol ethyl ester; and excipients or fillers as described herein.

In another embodiment, the compositions of the present invention comprise: an oxidative fat metabolizer which is L-carnitine; a neurotransmitter which is gamma amino butyric acid; an algin or algin equivalent from kelp extract; a medium chain triglyceride from coconut oil; and optionally at least one of the following: phosphatidylcholine, inositol and ethanolamine; turmeric; beeswax; gelatin; glycerol from palm fruit; one or more glycerol ethyl ester; excipients or fillers as described herein; and combinations thereof which may include any or all of the optional ingredients.

In another embodiment, the compositions of the present invention comprise: an oxidative fat metabolizer, e.g. L-carnitine, which is present in the composition in an amount of from about 10% to about 20%, from about 11% to about 19%, from about 12% to about 18%, from about 13% to about 17%, from about 14% to about 16%, and including, but not limited to, all ranges and subranges therebetween; a neurotransmitter, e.g. gamma amino butyric acid, which is present in the composition in an amount of from about 5% to about 25%, from about 6% to about 24%, from about 7% to about 23%, from about 8% to about 22%, from about 9% to about 21%, from about 10% to about 20%, from about 11% to about 19%, from about 12% to about 18%, from about 13% to about 17%, from about 14% to about 16%, and including, but not limited to, all ranges and subranges therebetween; an algin or algin equivalent, e.g. from kelp extract, which is present in the composition in an amount of from about 2% to about 5%, from about 2.5% to about 4.5%, from about 3.0% to about 4.0%, from about 3.5% to about 4.0%, and including, but not limited to, all ranges and subranges therebetween; a medium chain triglyceride, e.g. from coconut oil, which is present in the composition in an amount of from about 25% to about 45%, from about 26% to about 44%, from about 27% to about 43%, from about 28% to about 42%, from about 29% to about 41%, from about 30% to about 40%, from about 31% to about 39%, from about 32% to about 38%, from about 33% to about 37%, from about 34% to about 36%, and including, but not limited to, all ranges and subranges therebetween, of the composition; and optionally, at least one of the following: phosphatidylcholine, inositol and ethanolamine in a combined amount of from about 2% to about 15%, from about 3% to about 14%, from about 4% to about 13%, from about 5% to about 12%, from about 6% to about 11%, from about 7% to about 10%, from about 8% to about 9%, and including, but not limited to, all ranges and subranges therebetween; optionally turmeric in an amount of from about 0.1% to about 1.0%, from about 0.2% to about 0.9%, from about 0.3% to about 0.8%, from about 0.4% to about 0.7%, from about 0.45% to about 0.65%, from about 0.5% to about 0.6%, and including, but not limited to, all ranges and subranges therebetween; optionally beeswax in an amount of from about 0.05% to about 0.5%, from about 0.1% to about 0.45%, from about 0.15% to about 0.40%, from about 0.2% to about 0.35%, from about 0.25% to about 0.30%, and including, but not limited to, all ranges and subranges therebetween; optionally gelatin in an amount of from about 15% to about 20%, from about 15.5% to about 19.5%, from about 16% to about 19%, from about 16.5% to about 18.5%, from about 17% to about 18%, and including, but not limited to, all ranges and subranges therebetween; optionally glycerol, e.g. from palm fruit, in an amount of from about 5% to about 15%, from about 6% to about 14%, from about 7% to about 13%, from about 8% to about 12%, from about 9% to about 11%, and including, but not limited to, all ranges and subranges therebetween; optionally one or more glycerol ethyl ester in an amount of from about 0.1% to about 1.0%, from about 0.2% to about 0.9%, from about 0.3% to about 0.8%, from about 0.4% to about 0.7%, from about 0.5% to about 0.6%, and including, but not limited to, all ranges and subranges therebetween; optionally water in an amount of from about 0.5% to about 2.0%, from about 0.6% to about 1.9%, from about 0.7% to about 1.8%, from about 0.8% to about 1.7%, from about 0.9% to about 1.6%, from about 1.0% to about 1.5%, from about 1.1% to about 1.4%, from about 1.2% to about 1.3%, and including, but not limited to, all ranges and subranges therebetween; optionally excipients or fillers; and combinations thereof which may include any or all of the optional ingredients.

In another embodiment, the compositions of the present invention comprise: an oxidative fat metabolizer, e.g. L-carnitine, which is present in the composition in an amount of from about 16% to about 17%, from about 16.1% to about 16.9%, from about 16.2% to about 16.8%, from about 16.3% to about 16.7%, from about 16.4% to about 16.6%, and including, but not limited to, all ranges and subranges therebetween, of the composition; a neurotransmitter, e.g. gamma amino butyric acid, which is present in the composition in an amount of from about 6% to about 7%, from about 6.1% to about 6.9%, from about 6.2% to about 6.8%, from about 6.3% to about 6.7%, from about 6% to about 6.6.4%, and including, but not limited to, all ranges and subranges therebetween; an algin or algin equivalent, e.g. from kelp extract, which is present in the composition in an amount of from about 3% to about 4%, from about 3.1% to about 3.9%, from about 3.2% to about 3.8%, from about 3.3% to about 3.7%, from about 3.4% to about 3.6%, and including, but not limited to, all ranges and subranges therebetween; a medium chain triglyceride, e.g. from coconut oil, which is present in the composition in an amount of from about 26% to about 28%, from about 26.2% to about 27.8%, from about 26.4% to about 27.6%, from about 26.4% to about 27.6%, from about 26.6% to about 27.4%, from about 26.8% to about 27.2%, from about 26.9% to about 27.1%, and including, but not limited to, all ranges and subranges therebetween; and optionally at least one of the following: phosphatidylcholine, inositol and ethanolamine in a combined amount of from about 13% to about 14%, from about 13.1% to about 13.9%, from about 13.2% to about 13.8%, from about 13.3% to about 13.7%, from about 13.4% to about 13.6%, and including, but not limited to, all ranges and subranges therebetween; turmeric in an amount of from about 0.3% to about 0.5%, from about 0.32% to about 0.48%, from about 0.34% to about 0.46%, from about 0.36% to about 0.44%, from about 0.38% to about 0.42%, from about 0.39% to about 0.41%, and including, but not limited to, all ranges and subranges therebetween; optionally beeswax in an amount of from about 0.06% to about 0.07%, from about 0.061% to about 0.069%, from about 0.062% to about 0.068%, from about 0.063% to about 0.067%, from about 0.064% to about 0.066%, and including, but not limited to, all ranges and subranges therebetween, of the composition; optionally gelatin in an amount of from about 16% to about 17%, from about 16.1% to about 16.9%, from about 16.2% to about 16.8%, from about 16.3% to about 16.7%, from about 16.4% to about 16.6%, and including, but not limited to, all ranges and subranges therebetween; optionally glycerol, e.g. from palm fruit, in an amount of from about 13% to about 14%, from about 13.1% to about 13.9%, from about 13.2% to about 13.8%, from about 13.3% to about 13.7%, from about 13.4% to about 13.6%, and including, but not limited to, all ranges and subranges therebetween; optionally one or more glycerol ethyl ester in an amount of from about 0.3% to about 0.5%, from about 0.32% to about 0.48%, from about 0.34% to about 0.46%, from about 0.36% to about 0.44%, from about 0.38% to about 0.42%, from about 0.39% to about 0.41%, and including, but not limited to, all ranges and subranges therebetween; optionally water in an amount of from about 0.6% to about 0.7%, from about 0.61% to about 0.69%, from about 0.62% to about 0.68%, from about 0.63% to about 0.67%, from about 0.64% to about 0.66%, and including, but not limited to, all ranges and subranges therebetween; optionally an excipient or filler as described herein; and combinations thereof which may include any or all of the optional ingredients.

In another embodiment, the invention encompasses compositions comprising: an oxidative fat metabolizer, e.g., L-carnitine, and which is present in the composition in an amount of from about 10% to about 20%, from about 11% to about 19%, from about 12% to about 18%, from about 13% to about 17%, from about 14% to about 16%, and including, but not limited to, all ranges and subranges therebetween; a neurotransmitter, e.g., gamma amino butyric acid, which is present in the composition in an amount of from about 5% to about 25%, from about 6% to about 24%, from about 7% to about 23%, from about 8% to about 22%, from about 9% to about 21%, from about 10% to about 20%, from about 11% to about 19%, from about 12% to about 18%, from about 13% to about 17%, from about 14% to about 16%, and including, but not limited to, all ranges and subranges therebetween; an algin or algin equivalent, e.g., from kelp extract, which is present in the composition in an amount of from about 2% to about 5%, from about 2.5% to about 4.5%, from about 3.0% to about 4.0%, from about 3.5% to about 4.0%, and including, but not limited to, all ranges and subranges therebetween; a medium chain triglyceride, e.g., from coconut oil, which is present in the composition in an amount of from about 25% to about 45%, from about 26% to about 44%, from about 27% to about 43%, from about 28% to about 42%, from about 29% to about 41%, from about 30% to about 40%, from about 31% to about 39%, from about 32% to about 38%, from about 33% to about 37%, from about 34% to about 36%, and including, but not limited to, all ranges and subranges therebetween; optionally phosphatidylcholine, inositol and ethanolamine in a combined amount of from about 2% to about 15%, from about 3% to about 14%, from about 4% to about 13%, from about 5% to about 12%, from about 6% to about 11%, from about 7% to about 10%, from about 8% to about 9%, and including, but not limited to, all ranges and subranges therebetween; optionally turmeric in an amount of from about 0.1% to about 1.0%, from about 0.2% to about 0.9%, from about 0.3% to about 0.8%, from about 0.4% to about 0.7%, from about 0.45% to about 0.65%, from about 0.5% to about 0.6%, and including, but not limited to, all ranges and subranges therebetween; optionally beeswax in an amount of from about 0.05% to about 0.5%, from about 0.1% to about 0.45%, from about 0.15% to about 0.40%, from about 0.2% to about 0.35%, from about 0.25% to about 0.30%, and including, but not limited to, all ranges and subranges therebetween; optionally gelatin in an amount of from about 15% to about 20%, from about 15.5% to about 19.5%, from about 16% to about 19%, from about 16.5% to about 18.5%, from about 17% to about 18%, and including, but not limited to, all ranges and subranges therebetween; optionally glycerol, e.g., from palm fruit, in an amount of from about 5% to about 15%, from about 6% to about 14%, from about 7% to about 13%, from about 8% to about 12%, from about 9% to about 11%, and including, but not limited to, all ranges and subranges therebetween; optionally one or more glycerol ethyl ester in an amount of from about 0.1% to about 1.0%, from about 0.2% to about 0.9%, from about 0.3% to about 0.8%, from about 0.4% to about 0.7%, from about 0.5% to about 0.6%, and including, but not limited to, all ranges and subranges therebetween; optionally water in an amount of from about 0.5% to about 2.0%, from about 0.6% to about 1.9%, from about 0.7% to about 1.8%, from about 0.8% to about 1.7%, from about 0.9% to about 1.6%, from about 1.0% to about 1.5%, from about 1.1% to about 1.4%, from about 1.2% to about 1.3%, and including, but not limited to, all ranges and subranges therebetween; and optionally an excipient or filler as described herein.

In another embodiment, the invention encompasses compositions comprising: an oxidative fat metabolizer, e.g. L-carnitine, which is present in the composition in an amount of from about 16% to about 17%, from about 16.1% to about 16.9%, from about 16.2% to about 16.8%, from about 16.3% to about 16.7%, from about 16.4% to about 16.6%, and including, but not limited to, all ranges and subranges therebetween; a neurotransmitter, e.g. gamma amino butyric acid, which is present in the composition in an amount of from about 6% to about 7%, from about 6.1% to about 6.9%, from about 6.2% to about 6.8%, from about 6.3% to about 6.7%, from about 6% to about 6.6.4%, and including, but not limited to, all ranges and subranges therebetween; an algin or algin equivalent, e.g. from kelp extract, which is present in the composition in an amount of from about 3% to about 4%, from about 3.1% to about 3.9%, from about 3.2% to about 3.8%, from about 3.3% to about 3.7%, from about 3.4% to about 3.6%, and including, but not limited to, all ranges and subranges therebetween; a medium chain triglyceride, e.g. from coconut oil, which is present in the composition in an amount of from about 26% to about 28%, from about 26.2% to about 27.8%, from about 26.4% to about 27.6%, from about 26.4% to about 27.6%, from about 26.6% to about 27.4%, from about 26.8% to about 27.2%, from about 26.9% to about 27.1%, and including, but not limited to, all ranges in between of the composition; optionally phosphatidylcholine, inositol and ethanolamine in a combined amount of from about 13% to about 14%, from about 13.1% to about 13.9%, from about 13.2% to about 13.8%, from about 13.3% to about 13.7%, from about 13.4% to about 13.6%, and including, but not limited to, all ranges and subranges therebetween; turmeric in an amount of from about 0.3% to about 0.5%, from about 0.32% to about 0.48%, from about 0.34% to about 0.46%, from about 0.36% to about 0.44%, from about 0.38% to about 0.42%, from about 0.39% to about 0.41%, and including, but not limited to, all ranges and subranges therebetween; optionally beeswax in an amount of from about 0.06% to about 0.07%, from about 0.061% to about 0.069%, from about 0.062% to about 0.068%, from about 0.063% to about 0.067%, from about 0.064% to about 0.066%, and including, but not limited to, all ranges and subranges therebetween; optionally gelatin in an amount of from about 16% to about 17%, from about 16.1% to about 16.9%, from about 16.2% to about 16.8%, from about 16.3% to about 16.7%, from about 16.4% to about 16.6%, and including, but not limited to, all ranges and subranges therebetween; optionally glycerol, e.g. from palm fruit, in an amount of from about 13% to about 14%, from about 13.1% to about 13.9%, from about 13.2% to about 13.8%, from about 13.3% to about 13.7%, from about 13.4% to about 13.6%, and including, but not limited to, all ranges and subranges therebetween; optionally one or more glycerol ethyl ester in an amount of from about 0.3% to about 0.5%, from about 0.32% to about 0.48%, from about 0.34% to about 0.46%, from about 0.36% to about 0.44%, from about 0.38% to about 0.42%, from about 0.39% to about 0.41%, and including, but not limited to, all ranges and subranges therebetween; optionally water in an amount of from about 0.6% to about 0.7%, from about 0.61% to about 0.69%, from about 0.62% to about 0.68%, from about 0.63% to about 0.67%, from about 0.64% to about 0.66%, and including, but not limited to, all ranges and subranges therebetween; and optionally an excipient or filler as described herein.

In another embodiment, the invention encompasses compositions comprising an oxidative fat metabolizer, a neurotransmitter, an algin or algin equivalent, a medium chain triglyceride, phosphatidylcholine, inositol, ethanolamine, turmeric, beeswax, gelatin, water and vegetable glycerine.

In another embodiment, the compositions of the present invention comprise an oxidative fat metabolizer, a neurotransmitter, an algin or algin equivalent, a medium chain triglyceride, phosphatidylcholine, inositol, ethanolamine, turmeric, beeswax, gelatin, water and vegetable glycerine; wherein:

the oxidative fat metabolizer is carnitine; and the carnitine is in one embodiment L-carnitine;

the neurotransmitter is gamma amino butyric acid;

the algin or algin equivalent is kelp extract; and

optionally, the composition further comprises an excipient or filler as described herein. However, the compositions can be used alone without an excipient or filler.

In another embodiment, the compositions are suitable for oral administration. In another embodiment, the compositions of the present invention are in the form of a softgel capsule.

In another embodiment, the invention encompasses compositions comprising an oxidative fat metabolizer, a neurotransmitter, an algin or algin equivalent, a medium chain triglyceride, phosphatidylcholine, inositol, ethanolamine, turmeric, beeswax, gelatin, water and vegetable glycerine; wherein:

the oxidative fat metabolizer is present in the composition in an amount of from about 10% to about 20%;

the neurotransmitter is present in the composition in an amount of from about 5% to about 25%;

the algin or algin equivalent is present in the composition in an amount of from about 2% to about 5%;

the MCT is present in the composition in an amount of from about 25% to about 45%;

the phosphatidylcholine, inositol and ethanolamine are present in the composition in a combined amount of from about 2% to about 15%;

the turmeric is present in the composition in an amount of from about 0.1% to about 1%;

the beeswax is present in the composition in an amount of from about 0.05% to about 0.5%;

the gelatin is present in the composition in an amount of from about 15% to about 20%;

the vegetable glycerine is present in the composition in an amount of from about 5% to about 15%.

In another embodiment, the compositions of the present invention comprise of the present invention comprise: an oxidative fat metabolizer which is L-carnitine and is present in the composition in an amount of from about 16% to about 17%; a neurotransmitter which is gamma amino butyric acid and is present in the composition in an amount of from about 6% to about 7%; an algin or algin equivalent from kelp extract which is present in the composition in an amount of from about 3% to about 4%; a medium chain triglyceride from coconut oil which is present in the composition in an amount of from about 26% to about 28%; phosphatidylcholine, inositol and ethanolamine in a combined amount of from about 13% to about 14%; turmeric in an amount of from about 0.3% to about 0.5%; beeswax in an amount of from about 0.06% to about 0.07%; gelatin in an amount of from about 16% to about 17%; glycerol from palm fruit in an amount of from about 13% to about 14%; glycerol ethyl ester in an amount of from about 0.3% to about 0.5%; and water in an amount of from about 0.6% to about 0.7%.

In another embodiment, the compositions of the present invention comprise L-carnitine, gamma amino butyric acid, algin or algin equivalents from kelp extract, medium chain triglycerides from coconut oil, phosphatidylcholine, turmeric, beeswax, gelatin, vegetable glycerine from palm fruit and optionally excipients or fillers as described herein.

The compositions of the invention are useful in regulating body metabolism. The invention encompasses methods of regulating disorders associated with a deficiency in proper metabolism.

The invention further encompasses methods for regulating a condition in an animal, for example a mammal, for example a human, comprising administering to a mammal an effective amount of a composition comprising L-carnitine, GABA, kelp extract, MCT (for example, from coconut oil), and optionally phosphatidylcholine, inositol, ethanolamine, turmeric, beeswax, gelatin, glycerine (from, for example, palm fruit), glycerol ethyl ester, water, excipients/fillers (as described herein) and combinations thereof which may include any or all of the optional ingredients.

In another embodiment, the compositions of the present invention are useful for regulating serum HDL and LDL levels, for example increasing healthy HDL levels while decreasing unhealthy LDL levels. In another embodiment, the compositions of the present invention are useful for regulating serum triglyceride levels, for example by increasing healthy triglyceride levels. In another embodiment, the compositions of the present invention are useful for maintaining fat metabolism. In another embodiment, the compositions of the present invention are useful for maintaining healthy weight. In another embodiment, the compositions of the present invention are useful for maintaining memory and attention span. In another embodiment, the compositions of the present invention are useful for maintaining mood and mental stability. In another embodiment, the compositions of the present invention are useful for maintaining energy production with less risk of hypoglycemia. In another embodiment, the compositions of the present invention are useful for maintaining heart muscle function and heartbeat regularity. In another embodiment, the compositions of the present invention are useful for maintaining resilience. In another embodiment, the compositions of the present invention are useful for maintaining sperm health, motility and function.

The invention also encompasses compositions for topical use, e.g. compositions intended for use on a joint. For example, topical compositions include those described herein. The topical composition are useful in treating arthritis, rheumatoid and osteoarthritis; sports injuries; contusions; degenerative joint changes; and are useful for facilitated growth of artificial joint replacement components.

The compositions of the invention may be administered by any convenient route, for example, orally, topically, by intravenous infusion or bolus injection, by absorption through epithelial or mucocutaneous linings (e.g., oral mucosa, rectal and intestinal mucosa, etc.) and may be administered together with another biologically active agent.

The invention also encompasses kits for regulating a condition in a mammal comprising a container comprising one or more oxidative fat metabolizers, a neurotransmitter, and algin or algin equivalent, a medium chain triglyceride, and optionally, one or more pharmaceutically acceptable excipients/fillers, and instructions for use.

In another embodiment, the kits of the present invention comprise a container comprising the following components: L-carnitine, GABA, kelp extract, MCT from coconut oil, phosphatidylcholine, inositol, ethanolamine, turmeric, beeswax, gelatin, and glycerine from palm fruit, glycerol ethyl ester, water, instructions for use and combination thereof which may include any or all of the optional ingredients, wherein the each of the components is pre-measured into a respective unit of use amount.

In another embodiment, the compositions of the invention are dietary compositions.

### Oxidative Fat Metabolizers of the Invention

The invention encompasses compositions comprising one or more oxidative fat metabolizers. The oxidative fat metabolizers of the compositions of the present invention can be an amino acids, for example, glutamine and arginine. In another embodiment, the compositions of the invention comprising glutamine, arginine or combinations thereof enhance the immune system. In another embodiment, the compositions of the invention also promote anabolic activity (i.e., building of lean muscle mass) while glutamine buffers lactic acid buildup (causes muscle burn) to reduce fatigue. Glutamine, arginine and the branched chain amino acids (leucine, isoleucine and valine) are non-limiting examples of oxidative fat metabolizers suitable for use in the compositions of the present invention.

In another embodiment of the compositions of the present invention, the oxidative fat metabolizer is carnitine. Carnitine, for example L-carnitine, is a naturally occurring compound manufactured in the body from the amino acids lysine and methionine. While present in all tissues, it is found in muscle, heart and brain at higher levels. Compositions containing carnitine have beneficial effects on the human body when ingested from dietary sources. Carnitine is considered to be a health-enhancing substance that falls into the semi-essential category, meaning that it plays an important role in optimum health and longevity but it is not absolutely necessary to supplement with this substance for survival. Carnitine is used to move fuel sources into, and waste products out of, cells.

Primary carnitine deficiency is a condition that prevents the body from using fats for energy, particularly during periods without food. Without carnitine, fats cannot be processed correctly and are not converted into energy, which can lead to characteristic signs and symptoms of this disorder. People with primary carnitine deficiency have defective proteins called carnitine transporters, which bring carnitine into cells and prevent its escape from the body.

Typically, initial signs and symptoms of this disorder occur during infancy or early childhood and often include brain function abnormalities (encephalopathy); an enlarged, poorly pumping heart (cardiomyopathy); confusion; vomiting; muscle weakness; and low blood sugar (hypoglycemia). Serious complications such as heart failure, liver problems, coma, and sudden unexpected death are also a risk. Acute illness due to primary carnitine deficiency can be triggered by periods of fasting or illnesses such as viral infections, particularly when eating is reduced.

Primary carnitine deficiency is sometimes diagnosed in adults and is then thought to be less severe both in symptoms and life expectation. Treatment is usually done by supplementation of L-carnitine after assessing the severity of the deficiency after a muscular biopsy.

A deficiency in carnitine has also been linked to low sperm motility in some men. Carnitine and acetylated carnitine (L-acetylcarnitine) are found in high concentrations in the epididymis, where they also act as antioxidants, protecting spermatozoa against damage caused by reactive oxygen species. Investigation of the link between seminal carnitine levels and spermatozoal function, and the effect of combined L-carnitine+L-acetylcarnitine therapy, in infertile men identified a significant correlation between seminal carnitine concentration and several key markers of sperm health and function. Therefore, L-carnitine/L-acetylcarnitine treatment may be an effective therapy to improve sperm motility and function (De Rosa et al., Drugs R.D. 6:1-9 (2005)).

Obesity and type 2 diabetes are characterized by impaired vascular endothelial function, an early step in the development of atherosclerotic disease. Elevated free fatty acid levels, decreased free fatty acid oxidation, and decreased carnitine levels characterize obesity and type 2 diabetes. As carnitine has been reported to exhibit vasoprotective properties, it may alleviate free fatty acid induced vascular dysfunction. In lean and obese individuals, oral carnitine supplementation exerted protective effects on the vasculature as measured by improved leg blood flow (Steinberg, “L-carnitine Ameliorates Vascular Dysfunction Caused by Elevated Free Fatty Acids,” two-day conference held Mar. 25-26, 2004 at the Lister Hill Auditorium in Bethesda, Md., 2004).

A deficiency in carnitine has also been implicated in various conditions including: cirrhosis of the liver, memory loss, depression, recurrent infections, respiratory distress in infants, fatigue, depression, heart problems, weakness, hypoglycemia, fat accumulation, heart disease, angina and other ailments. This deficiency may be made worse by consuming alcohol, fatty foods, and sugar.

Carnitine may also increase endurance and exercise tolerance (Marconi et al., Eur. J. Appl. Physiol. 54(2):131-135 (1985)). Supplementation of a person's diet with carnitine may help to relieve symptoms associated muscle weakness and fatigue. It may also alleviate an inability to reach peak exercise goals, decrease recovery time after exercise, and has been tied to improved performance of seasoned athletes.

Carnitine is also concentrated in cardiac muscle, which uses fatty acids as its primary fuel and supplementation may help to improve cardiac arrhythmia, congestive heart failure and cardiomyopathy, as well as recovery from a heart attack or bypass surgery. It has been shown to decrease the severity of a heart attack and to improve exercise tolerance, including walking distance, in those who suffer from angina and poor circulation. It can protect the heart from the toxic effects of chemotherapy known to damage the heart and even cause death from heart damage. Studies show that carnitine can reduce myocardial injury after ischemia and reperfusion by counteracting the toxic effects of free fatty acids and improving carbohydrate metabolism. In short-term studies, carnitine has been shown to have anti-ischemic properties. Studies have shown that administration of intravenous and oral carnitine at relatively high amounts reduced mortality and heart failure (Ferrari, “Therapeutic Effects of L-carnitine and Propionyl-L-carnitine on Cardiovascular Diseases: A Review,” two-day conference held Mar. 25-26, 2004 at the Lister Hill Auditorium in Bethesda, Md., 2004).

In addition, carnitine might play a role in hypertriglyceridemia. Carnitine, which is necessary for fatty acid oxidation, has been reported to lower serum triglycerides in patients with type IV hyperlipoproteinemia. Results of other studies suggest that carnitine may be effective in the treatment of hypertriglyceridemia in patients of hemodialysis with the only reported side effect being a sense of euphoria. (Guarnieri et al., Am. J. Clin. Nutr. 33:1489-1492 (1980)). Consumption of supplementary carnitine has also been linked to a significant drop in triglycerides, serum lipids, and cholesterol (Abdel-Aziz et al., Nutr. Rep. Internat. 29:1071 (1984), Maebashi et al., Lancet 2(8094):805-807 (1978) and Bougneres et al., Lancet 1(8131):1401-2 (1979)).

In addition to aiding in raising HDL levels, carnitine may stimulate nerve cells to enhance acetylcholine (the primary neurotransmitter of the brain) production (Science News Nov. 30, 1991, pg 365) as well as mimic the actions of acetylcholine. These effects may help improve memory, attention span, senility, learning disabilities and brain-blood flow.

Separate studies have investigated the effects of carnitine supplementation on memory, attention and other aspects of mental health. Accumulation of oxidative damage to mitochondria, protein, and nucleic acid in the brain may lead to various neuronal and cognitive dysfunctions. Supplementation with carnitine has been shown to reverse some of these effects (Liu et al., PNAS 99:2356-2361 (2002)). These reversing effects have also been observed in severe disorders such as Alzheimer's disease (Bianchetti et al., Curr. Med. Res. Opin. 19:350-353 (2003)).

Carnitine may also play a role in regulating glucose metabolism. Research studies indicate that carnitine stimulates glucose disposal and oxidation (De Gaetano et al., J. Am. Coll. Nutr. 18:289-295 (1999)). Separate studies provide direct evidence that carnitine can stimulate glucose oxidation in the intact fatty acid perfused heart (Broderick et al., J. Biol. Chem. 267:3758-3763 (1992)).

The compositions of the invention are also useful in regulating carnitine in elderly people, as well as for people with metabolic carnitine deficiency condition; a condition in which the body does not produce enough carnitine to meet its metabolic demands.

The arginine, carnitine, or glutamine or other fat metabolizers may be used as acceptable salts or acceptable prodrugs thereof. The amino acids used in the compositions of the invention can be D, L, or mixtures thereof. The L-form of fat metabolizers is an example of the fat metabolizers utilized in the compositions of the invention.

The phrase “acceptable salt(s)” as used herein includes but is not limited to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds, included in the present compositions, that are acidic in nature are capable of forming base salts with various pharmacologically acceptable cations. Examples of such salts include alkali metal or alkaline earth metal salts and, calcium, magnesium, sodium lithium, zinc, potassium, and iron salts.

As used herein and unless otherwise indicated, the term “acceptable prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise oligonucleotides, peptides, lipids, aliphatic and aromatic groups, or NO, NO₂, ONO, and ONO₂ moieties. Prodrugs can typically be prepared using well known methods, such as those described in Burger's Medicinal Chemistry and Drug Discovery, pp. 172, 178, 949, 982 (Manfred E. Wolff ed., 5th ed. 1995), and Design of Prodrugs (H. Bundgaard ed., Elselvier, N.Y. 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxy-methyl, pivaloyloxymethyl, and pivaloyloxyethyl esters), lactonyl esters (such as phthalidyl and thiophthalidyl esters), lower alkoxyacyloxyalkyl esters (such as methoxycarbonyloxy-methyl, ethoxycarbonyloxy-ethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, a amino acid amides, alkoxyacyl amides, and alkylaminoalkyl-carbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

### Neurotransmitters of the Invention

Neurotransmitters are small signaling molecules that are released in response to stimuli and in turn mediate communication between neurons. According to the prevailing beliefs of the 1960s, a chemical can be classified as a neurotransmitter if it meets the following conditions:

it is synthesized endogenously (within the presynaptic neuron); it is available in sufficient quantity in the presynaptic neuron to exert an effect on the postsynaptic neuron; externally administered, it must mimic the endogenously-released substance; and a biochemical mechanism for inactivation must be present. However, there are other materials, such as the zinc ion, that are neither synthesized nor catabolized and are considered neurotransmitters by some.

Substances that act as neurotransmitters can be roughly categorized into three major groups: (1) amino acids (primarily glutamic acid, GABA, aspartic acid & glycine), (2) peptides (vasopressin, somatostatin, neurotensin, etc.) and (3) monoamines (norepinephrine NA, dopamine DA & serotonin 5-HT) plus acetylcholine (ACh). The major neurotransmitters of the brain are glutamic acid (glutamate) and GABA. Neurotransmitters can be broadly classified into small-molecule transmitters and neuroactive peptides. Around 10 small-molecule neurotransmitters are known: acetylcholine, 5 amines, and 3 or 4 amino acids (depending on exact definition used), Purines, (Adenosine, ATP, GTP and their derivatives) are neurotransmitters. Fatty acids are also receiving attention as the potential endogenous cannabinoid. Over 50 neuroactive peptides have been found, among them hormones such as LH or insulin that have specific local actions in addition to their long-range signaling properties. Single ions, such as synaptically-released zinc, are also considered neurotransmitters by some.

Within the cells, small-molecule neurotransmitter molecules are usually packaged in vesicles. When an action potential travels to the synapse, the rapid depolarization causes calcium ion channels to open. Calcium then stimulates the transport of vesicles to the synaptic membrane; the vesicle and cell membrane fuse, leading to the release of the packaged neurotransmitter, a mechanism called exocytosis.

The neurotransmitters then diffuse across the synaptic cleft to bind to receptors. The receptors are broadly classified into ionotropic and metabotropic receptors. Ionotropic receptors are ligand-gated ion channels that open or close through neurotransmitter binding. Metabotropic receptors, which can have a diverse range of effects on a cell, transduct the signal by secondary messenger systems, or G-proteins.

Neuroactive peptides are made in the neuron's soma and are transported through the axon to the synapse. They are usually packaged into dense-core vesicles and are released through a similar, but metabolically distinct, form of exocytosis used for small-molecule synaptic vesicles.

A neurotransmitter's effect is determined by its receptor. For example, GABA can act on both rapid or slow inhibitory receptors (the GABA-A and GABA-B receptor respectively). Many other neurotransmitters, however, may have excitatory or inhibitory actions depending on which receptor they bind to.

Neurotransmitters may cause either excitatory or inhibitory post-synaptic potentials. That is, they may help the initiation of a nerve impulse in the receiving neuron, or they may discourage such an impulse by modifying the local membrane voltage potential. In the central nervous system, combined input from several synapses is usually required to trigger an action potential. Glutamate is the most prominent of excitatory transmitters; GABA and glycine are well-known inhibitory neurotransmitters.

Many neurotransmitters are removed from the synaptic cleft by a process called reuptake (or often simply uptake). Without reuptake, the molecules might continue to stimulate or inhibit the firing of the postsynaptic neuron. Another mechanism for removal of a neurotransmitter is digestion by an enzyme. For example, at cholinergic synapses (where acetylcholine is the neurotransmitter), the enzyme acetylcholinesterase breaks down the acetylcholine. Neuroactive peptides are often removed from the cleft by diffusion, and eventually broken down by proteases.

While some neurotransmitters (glutamate, GABA, glycine) are used very generally throughout the central nervous system, others can have more specific effects, such as on the Autonomic nervous system, by both pathways in the sympathetic nervous system and the parasympathetic nervous system, and the action of others are regulated by distinct classes of nerve clusters which can be arranged in lamilar pathways around the brain. For example, Serotonin is released specifically by cells in the brainstem, in an area called the raphe nuclei, but travels around the brain along the medial forebrain bundle activating the cortex, hippocampus, thalamus, hypothalamus and cerebellum. Also, it is released in the Caudal sertonin nucli, so as to have effect on the spinal cord. In the peripheral nervous system (such as in the gut wall) serotonin regulates vascular tone. Dopamine classically modulates two systems: the brain's reward mechanism, and movement control.

Neurotransmitters that have these types of specific actions are often targeted by drugs. Cocaine, for example, blocks the reuptake of dopamine, leaving these neurotransmitters in the synaptic gap longer. Prozac is a serotonin reuptake inhibitor, hence potentiating its effect. AMPT prevents the conversion of tyrosine to L-DOPA, the precursor to dopamine; reserpine prevents dopamine storage within vesicles; and deprenyl inhibits monoamine oxidase (MAO)-B and thus increases dopamine levels.

Some neurotransmitter/neuromodulators like zinc not only can modulate the sensitivity of a receptor to other neurotransmitters (allosteric modulation) but can even penetrate specific, gated channels in post-synaptic neurons, thus entering the post-synaptic cells. This “translocation” is another mechanism by which synaptic transmitters can affect postsynaptic cells.

Diseases may affect specific neurotransmitter pathways. For example, Parkinson's disease is at least in part related to failure of dopaminergic cells in deep-brain nuclei, for example the substantia nigra. Treatments potentiating the effect of dopamine precursors have been proposed and effected, with moderate success.

The compositions of the invention also comprise one or more neurotransmitters, in one embodiment GABA. The neurotransmitters of the invention are useful in that they stabilize neurochemical communications, stabilize cell metabolism, provide endurance, and resilience and promote efficacious cell metabolism.

Non-limiting examples of neurotransmitters of the invention include 5-hydroxytryptamine (5-HT), tryptophan, gamma amino butyric acid (“GABA,” γ-aminobutyrate, 4-aminobutyrate), glutamate, aspartate, glycine, histamine, histidine, epinephrine, tyrosine, norepinephrine and combinations thereof.

GABA, or gamma-aminobutyric acid, is the most abundant inhibitory neurotransmitter in the brain and is also a well-known inhibitor of presynaptic transmission in the retina. While GABA is an amino acid, it is classified as a neurotransmitter and helps induce relaxation and sleep by inhibiting over-excitation of neurons. GABA contributes to motor control, vision, cortical functions, and the regulation of anxiety related responses.

Gamma-aminobutyric acid also stimulates the anterior pituitary, leading to higher levels of Human Growth Hormone (HGH). Human Growth Hormone contributes significantly to muscle growth and also prevents the creation of fat cells. Moreover, HGH depletion may contribute to sleep disorders.

GABA exerts its effects by binding to two distinct receptors, GABA-A and GABA-B. The GABA-A receptors form a Cl⁻ channel. The binding of GABA to GABA-A receptors increases the Cl⁻ conductance of presynaptic neurons. The anxiolytic drugs of the benzodiazepine family exert their soothing effects by potentiating the responses of GABA-A receptors to GABA binding. The GABA-B receptors are coupled to an intracellular G-protein and act by increasing conductance of an associated K⁺ channel.

Algin or Algin Equivalents

The compositions of the invention also comprise one or more algins or equivalents thereof. As used herein, “algins or equivalents thereof” include, algiline, algin (laminaria spp. and other kelps), algin (polysaccharide), alginate kmf, alginic acid, sodium salt, algipon l-1168, amnucol, antimigrant c 45, cecalgine tbv, cohasalih, darid qh, dariloid qh, duckalgin, fema no. 2014, hsdb 1909, halltex, kelco gellv, kelcosol, kelgin, kelgin f, kelgin hv, kelgin lv, kelgin xl, kelgum, kelp extract, kelset, kelsize, keltex, keltone, 1-algiline, lamitex, manucol, manucol dm, manucol kmf, manucol ss/ld21 manugel f 331, manutex, manutex f, manutex rs 1, manutex rs-5, manutex sa/kp, manutex sh/1 h, manutex rs1, meypralgin r/lv, minus, mosanon, nouralgine, og 1, pectalgine, proctin, protacell 8, protanal, protatek, snow algin h, snow algin l, snow algin m, sodium alginate (usan), sodium alginate, sodium polymannuronate, stipine, tagat, or tragaya or combinations thereof. In another embodiment, the algin is kelp extract, for example, high algine kelp or a functionally equivalent complex carbohydrate.

The algin of the invention contains carbohydrates, oils, proteins, vitamins, trace elements, minerals and fibers in balanced proportions. In another embodiment, the invention contains high levels of minerals (salts) and trace elements (metals), which are highly beneficial to human beings. These minerals and elements support thyroid hormone for better fat metabolism and enhanced energy. Alginate supports the thyroid and balances metabolism. Consequently, thyroid function is improved and healthy weight is easier to attain.

In one embodiment, algin is a gelatinous substance produced by brown algae, and is often used in food and pharmaceutical preparations. In another embodiment, algin offers especially good protection from many kinds of modern day pollutants, carcinogens, and toxins. In one embodiment, algin prevents living tissue from absorbing radioactive materials. In one embodiment, algin also encourages the action of dietary fiber, by supplying nutrients, and by normalizing bowel functions.

Chemically, algin is a linear copolymer with homopolymeric blocks of (1-4)-linked β-D-mannuronate (M) and its C-5 epimer α-L-guluronate (G) residues, respectively, covalently linked together in different sequences or blocks.

The monomers can appear in homopolymeric blocks of consecutive G-residues (G-blocks), consecutive M-residues (M-blocks), alternating M and G-residues (MG-blocks) or randomly organized blocks. The relative amount of each block type varies both with the origin of the alginate. Alternating blocks form the most flexible chains and are more soluble at lower pH than the other blocks. G-blocks form stiff chain elements, and two G-blocks of more than 6 residues each form stable cross-linked junctions with divalent cations (e.g. Ca²⁺, Ba²⁺, Sr²⁺ among others) leading to a three-dimensional gel network. At low pH, protonized alginates will form acidic gels. In these gels, it is mostly the homopolymeric blocks that form the junctions, where the stability of the gel is determined by the relative content of G-blocks.

Alginate strengthens mucus, the body's natural protection of the gut wall, can slow digestion down, and can slow the uptake of nutrients in the body (Pearson, Critical Reviews in Food Science and Nutrition 45(6):497-510 (September 2005)). Studies have shown that as few as 5 g of soluble fiber in the form of alginate significantly decreased the post-meal rise in glucose and insulin (Torsdottir et al., J. Nutr. 121(6):795-799 (1991)). Alginate may also enhance glycemic-control and lipid-lowering effects (Andallu et al., Clin. Chim. Acta. 314(1-2):47-53 (2001)). Algin in the diet, as from kelp (seaweed) or the supplement sodium alginate, helps to bind lead and other heavy metals and toxins in the gastrointestinal tract and enhances their elimination (Haas, Staying Healthy with Nutrition: The Complete Guide to Diet and Nutritional Medicine (2006), improves digestion, reduces toxin exposure to the kidney, increases circulation, and reduces toxic metabolites in the blood. Algins may also normalize low blood pressure, while normal and high blood pressure are unaffected.

### Medium-Chain Triglycerides (MCTs)

Medium-chain triglycerides, MCTs, are nonvolatile alkalinizing fatty acid esters of glycerol (e.g., medium-chain fatty acid esters of glycerol). Non-limiting examples of MCTs suitable for use in the compositions of the present invention are fatty acid esters of glycerol in which the fatty acid moieties thereof have from about 4 to about 16 carbon atoms, or about 6 to 12 carbon atoms, and in one embodiment, an average of about 8 carbon atoms. The fatty acid moieties of the MCTs of the present invention can be the same or different, and can be saturated or unsaturated.

MCTs suitable for use on the compositions of the present invention are those commonly found in coconut and palm kernel oils and are also found in camphor tree drupes. MCTs include coconut and palm kernel oils themselves, or extracts thereof.

It is preferable that the MCTs of the present invention are prepared by a relatively “mild” processing methods which do not denature or otherwise change the “native” characteristics of the MCT. For example, the MCTs of the present invention can be prepared under temperature-controlled conditions, e.g. at temperatures less than 80° F. The term “native” refers to the chemical and/or physical characteristics of the MCT in the unprocessed plant source (e.g. coconuts or palm kernels).

The physiology and biochemistry of medium-chain triglycerides are very different from those of long-chain triglycerides. Long-chain triglycerides are first hydrolyzed in the small intestine to long-chain fatty acids. They are in turn re-esterified in the mucosal cells of the small intestine to long-chain triglycerides, which are then carried by chylomicrons and transported via the lymphatic system to the systemic circulation. The systemic circulation in turn distributes the long-chain triglycerides to various tissues of the body, including adipose tissue and the liver.

MCTs are rapidly absorbed from the small intestine and transported to the liver. Since MCTs, in contrast with long-chain triglycerides, LCTs, do not require pancreatic enzymes or bile salts for digestion and absorption, MCTs are better handled in those with malabsorption syndromes than are the long-chain fatty acids. These syndromes include pancreatic disorders, hepatic disorders, gastrointestinal disorders and disorders of the lymph system (Yost et al., Am. J. Clin. Nutr. 49(2):326-330 (1989).

Medium-chain fatty acids are taken up by hepatocytes and converted to medium-chain fatty acyl CoA which enters mitochondria without requiring the aid of carnitine. On the other hand, long-chain fatty acids, which are also converted to their coenzyme A esters in cells, including hepatocytes, require that they be converted from coenzyme A esters to carnitine esters in order to be transported across the mitochondrial membrane. Within the hepatocyte mitochondria, medium-chain fatty acyl CoA is converted to acetoacetate and beta-hydroxybutyrate and subsequently to carbon dioxide, water and energy. The oxidation of MCT produces 8.3 kilocalories of energy per gram ingested.

MCTs are therefore easier to metabolize, which could be advantageous to those who are critically ill and those with carnitine deficiencies.

MCTs are ketogenic. The metabolism of MCT in hepatocytes produces two so-called ketone bodies, acetoacetate and beta-hydroxybutyrate. These ketone bodies are carried by the bloodstream to other tissues of the body, where they are used for energy production, as well as for other biochemical processes. It is believed that ketosis may raise the seizure threshold and reduce seizure severity. This is still hypothetical but is the rationale for the use of ketogenic diets in the treatment of seizure disorders.

MCTs are easier to metabolize are easily converted into usable sources of energy, and very rarely are converted and stored within the body as fat. Additionally, studies suggest a greater satiating effect of medium-chain triglycerides (MCT) compared with long-chain triglycerides (LCT), thereby facilitating weight control when included in the diet as a replacement for fats containing LCT (St-Onge et al., J. Nutr. 132:329-332 (2002)). Further studies suggest that consumption of MCTs increase energy expenditure (EE) (Scalfi et al., Am. J. Clin. Nutr. 53:1130-1133 (1991), Seaton et al., Am. J. Clin. Nutr. 44:630-634 (1986), Dulloo et al., Eur. J. Clin. Nutr. 50:152-158 (1996), Hill et al., Metabolism 38:641-648 (1989) and White et al., Am. J. Clin. Nutr. 69:883-889 (1999)) and result in decreased fat cell size and body weight accretion (Baba et al., Am. J. Clin. Nutr. 35:678-682 (1982), Crozier et al., Metabolism 36:807-814 (1987), Geliebter et al., Am. J. Clin. Nutr. 37:1-4 (1983), Lavau et al., J. Nutr. 108:613-620 (1978), Hill et al., Int. J. Obes. 17:223-236 (1993), Yost et al., Am. J. Clin. Nutr. 49:326-330 (1989) and Bray et al., Int. J. Obes. 4:27-32 (1980)).

Consumption of a diet rich in MCTs results in greater loss of adipose tissue compared with LCTs, perhaps due to increased energy expenditure and fat oxidation observed with MCT intake. Thus, MCTs may be considered as agents that aid in the prevention of obesity or potentially stimulate weight loss (St-Onge et al., Obesity Research 11:395-402 (2003)).

### Phosphatidylcholine, Inositol and Ethanolamine

Phosphatidylcholine is a phospholipid present in abundance in cell membranes, and actively participates in the structure and transport of molecules between the cells (Strayer et al., in Bioquimica, Third Edition. pp. 246-247 (1996)). Ethanolamine and inositol are precursors of phosphatidylethanolamine and phosphatidylinositol, respectively, and are also present in cell membranes, performing similar functions.

Phosphatidylcholine substances may alter cholesterol and other triglyceride metabolisms, increasing cholesterol solubility, altering the composition of fat deposits, and inhibiting plaque aggregation (Strayer et al., in Bioquimica, Third Edition. pp. 246-247 (1996)). For these reasons, phosphatidylcholine is used in the intravenous treatment of lipid atheromas, hypercholesterolemia, fat embolism, fatty deposits or plaque adhering to arterial walls, mental disturbances, hepatic and cardiac conditions induced by medication, alcohol, pollution, virus, and toxins (Navder et al., Life Sci. 61(19):1907-1914 (1997), Maranhao et al., Atherosclerosis 126(1):15-25 (1996), Bialecka, Ann. Acad. Med. Stetin. 43:41-56 (1997), Brook et al., Biochem. Med. Metab. Biol. 35(1):31-9 (1986), Melchinskaya et al., Terapevticheskii Arkhiv 72(8):57-58 (2000), Ozerova et al., Atherosclerosis 144(supl. 1):33 (1999), Polichetti et al., Life Sci. 67(21):2563-2576 (2000), Takahashi et al., Nutr. Sci. Vitaminol. 28(2):139-147 (1982) and Simonsson et al., Am. J. Clin. Nutr. 35(1):36-41 (1982)).

Some studies suggest that administration of phosphatidylcholine increases brain acetylcholine concentration and improves memory in mice with dementia (Chung et al., J. Nutr. 125:1484-1489 (1995)). Other studies in humans have shown significant improvement in explicit memory after ingestion of phosphatidylcholine (Ladd et al., Clin Neuropharmacol. 16(6):540-549 (1993)).

Inositol is classified as a member of the vitamin B complex, though it is not considered a vitamin itself because it can be synthesized by the human body. There are at least nine distinct isomers of inositol, and the terms for each are often used interchangeably including, but not limited to: inositol, myo-inositol, misoinositol, lipotropic factor, hexahydroxycyclohexane, cyclohexanehexyl, mouse antialopecia factor and, chemically, as cis-1,2,3,5-trans-4,6-cyclohexanehexyl. Inositol is involved in many biological processes, including: cytoskeleton assembly, nerve guidance, intracellular calcium (Ca2+) concentration control, cell membrane potential maintenance, serotonin activity modulation, breakdown of fats and reducing blood cholesterol and gene expression.

Studies have shown that administration of inositol is effective in depression, panic, and obsessive-compulsive disorder (Fux et al., Am. J. Psych. 153:1219-1221 (1996)). Others have shown beneficial effects of inositol in treating panic attacks and agoraphobia (Benjamin et al., Am. J. Pysch 152:1084-1086 (1995)).

Ethanolamine, also called 2-aminoethanol or monoethanolamine (often abbreviated as MEA), is an organic chemical compound which is both a primary amine (due to an amino group in its molecule) and a primary alcohol (due to a hydroxyl group). One study has demonstrated that chronic administration of a modified ethanolamine lead to up-regulation of GABA binding sites (Sykes et al., Biochem. Pharmacol. 33:387-393 (1984)), which would increase the effectiveness of GABA found in the embodiments of the invention. Another study has linked the proliferation of liver cells associated with toxic damage to the administration of ethanolamine (Murakami et al., 94:137-144 (1998)).

### Turmeric

Turmeric, also known as curcumin, displays antioxidative, anticarcinogenic and hypocholesterolemic activities. Studies have indicated that turmeric, ingested in the form of dietary curcuminoids, has lipid-lowering potency in vivo, probably due to alterations in fatty acid metabolism. (Asai et al., J. Nutr. 131(11):2932-2935 (2001)). Further studies suggest that oral administration of a nutritional dose of turmeric may reduce the susceptibility to oxidation of erythrocyte and liver microsome membranes in vitro and may contribute to the prevention of effects caused by a diet high in fat and cholesterol in blood and liver during the development of atherosclerosis (Mesa et al., Nutrition 19(9):800-804 (2003)). Additionally, recent studies have shown that the bioavailability of turmeric may be increased by formulation with phosphatidylcholine (Marczylo et al., Cancer Chemother. Pharmacol. 2006 Oct. 19; [Epub ahead of print]).

### Softgel Capsules

Softgel formulation characteristics consist of water or oil soluble fill solution, or suspension of drug covered by a layer of gelatin (made of gelatin, plasticizer, modifier, water, color, antioxidant or flavor). The outer layer can be enteric coated. The softgel delivery system offers improve d, rapid and consistent absorption of hydrophobic drugs.

The softgel delivery system is a unitary package, formed with gelatin outer layers, that contain between them the active ingredients in solution, suspension or paste form. The softgel capsule may have several shapes and sizes, dependent on the design.

Hydrophobic drugs can result in poor bio availability. These drugs will not dissolve readily in water, gastric or intestinal fluid and when they are compounded in solid dosage forms, the dissolution rate may be slow, absorption may vary and the bio availability may be incomplete. In the case of hydrochlorothiazide, isotretinoin and griseofulvin, bio availability is improved in the presence of fatty acids e.g. mono or diglycerides. Fatty acids can solubilize hydrophobic drugs in the gut and enable more rapid absorption. The softgel delivers drugs in solution and yet offers solid dosage form. Hydrophobic drugs are dissolved in hydrophilic solvent, which, when crushed or chewed, release the drug immediately to produce a solution of the drug in gastric juice ready for absorption from the gastrointestinal tract into the blood stream. This results in rapid onset of desired therapeutic effects. Acid soluble compounds may remain in solution and acid-insoluble compounds may precipitate as a fine particle cloud, but re dissolve quickly and give good bio availability results.

The development time for softgel is shorter due to lower bio-availability concerns and such solutions can be marketed at a fraction of cost. For example, Ibuprofen softgel gives rise to a shorter time to peak plasma concentration and greater peak plasma concentration compared to a marketed tablet formulation. Cyclosporin can give therapeutic blood levels which are not achievable from tablet form. Similarly oral hypoglycemic glipizide in softgel is also known to have better bio availability results compared with tablet form. Softgel delivery systems can also incorporate phospholipids or polymers or natural gums to entrap the drug active in the gelatin layer with an outer coating to give desired delayed/control release effects.

It is important that formulations of softgel fills have pH 2.5-7.5 otherwise hydrolysis or tanning can occur. The different acidic grades of gelatin blooms can be employed to address the problem of water migration and content greater than 20% will dissolve the capsule shell.

Softgel capsules are used beneficially in several industries including: the pharmaceutical, cosmetic, nutrition and veterinary industries.

The softgel capsule offers the following advantages over other oral delivery systems, such as hardshell capsules. Unitary one piece dosage, tight sealing in an automatic manner, easy to swallow, allow product identification (using colors and several shapes), allow uniformity, precision and accuracy between dosages, better stability than other oral delivery systems, good availability and rapid absorption, offer protection against contamination, light and oxidation are some of the advantages of a softgel capsule. Other advantages include: avoidance of unpleasant flavors due to content encapsulation, use in rectal, vaginal or ophthalmic drug delivery system due to a more forgiving shape, improved filling reproducibility, elegance and attractiveness as a finished product.

The shape and size of the capsule are defined depending on the needs of the product as well as the market. A number of possible softgel finished appearances and textures are possible: transparent/color, solid colors, transparent, solid colors in combination of two tones, transparent in two tones and transparent/solid colors.

Manufacturing soft gelatin capsules implicates the use of sophisticated technology. The rotary type softgel encapsulation process offers accuracy of dosage and higher production capacity. Before encapsulation process begins, gelatin mass for out shell and medicine for the capsule fill are prepared. The gelatin powder is mixed with water and glycerine, heated and stirred under vacuum. The outer layer of this special stainless steel vessel is steam-jacketed. Any required flavors or colors are added using a turbine mixer to molten gelatin and transferred to mobile vessels. The gelatin mass is kept in a steam-jacketed storage vessel at a constant temperature.

The medicine fill is prepared using standard procedures used in pharmaceutical liquid, paste or suspension manufacturing.

The encapsulation process begins when molten gel is pumped to the machine and two thin ribbons of gel (i.e. gelatin) are formed on either side of machine. These ribbons then pass over a series of rollers and over a set of dies that determine the size and shapes of capsules. The medicine fill is fed from its container to a positive displacement pump, which accurately doses the fill and injects it between two gelatin ribbons prior to sealing them together through the application of heat and pressure. The resulting capsules have the shape of an oblate oval, and have a seam where the two ribbons of gel are sealed together around the fill. The capsules formed at this stage are incredibly flexible due to water in gel mass. To remove excess water capsules pass through a conveyer into tumble dryers where about 25% of water is removed. The capsules are then placed on trays which are stacked and transferred into drying rooms where dry air is forced over capsules to remove any excess moisture. The moisture is measured at regular intervals, when the moisture is limited to approx. 8% the drying process is complete and capsules are ready for packaging.

The manufacturing of softgel delivery systems is carried out in a high productivity rotatory die machine and capsules are dried using an advanced tumble drier offering: dosage precision and accuracy, automation, easy cleaning and sanitation, high productivity, product variety and encapsulation in the absence of oxygen and/or light.

It is also possible to manufacture round seamless capsules (pearls) using a unique technology that allows manufacturing using the physical properties of superficial tension.

The productivity of a softgel capsule increases or diminishes upon considering the following variables: asset to encapsulate (density, consistency, etc.), capsule size and shape.

A number of compounds can be formulated to deliver faster onset of effect with lower dosage and lower side effects. Certain compounds could benefit from softgel formulation to give faster absorption, improved and uniform bio availability.

Softgel delivery systems also offer opportunities for many new chemical entities including peptides/other biopharmaceuticals and other pharmaceuticals those requiring reformulation due to bio-availability concerns.

Hitherto, it is not been practical to prepare softgel dosage forms of compositions containing quaternary amines such as carnitine. Quaternary amines are essentially aprotic solvents which tend to diffuse through and cause splitting or failure of the seam of the softgel capsule. The softgel dosage form of the compositions of the present invention solve this problem by modifying the softgel capsule itself, and the processing conditions used in forming the filled softgel capsule. Softgel capsules or provided in various standard sizes (e.g., 18, 20, etc.) and each standard size has characteristic dimensions and comprises a specified amount of gelatin. Softgel capsule dosage form of the present invention employs a modified “fat” 18 softgel structure, in which the amount of gelatin used for a size 20 softgel capsule is used for size 18 softgel capsule die which has been enlarged to retain the same interior volume of the capsule, despite the larger amount of gelatin used. The “fat” size 18 softgel capsule has the same size long axis as a standard size 18 softgel capsule, but is wider in its short axis. In addition, the softgel capsules of the present invention are manufactured under conditions in which the curing time for sealing the seam of the capsule is increased so that the seam essentially disappears (i.e., the gelatin from each half of the capsule mix with each other so completely that there is no readily discernible seam). This essentially eliminates leakage or diffusion of the carnitine component from the softgel during storage. In addition, emulsification of the carnitine on the other components of the formulation also servers to reduce leakage or diffusion from the softgel capsule.

Softgel capsules provide improved delivery of the components of the composition of the present invention compared to alternative dosage forms. For example, the uptake of L-carnitine by patients from tablets (e.g., measured by serum plasma levels) is about 10-14%; the uptake of L-carnitine from standard hard capsules is about 15-20%; whereas the uptake of L-carnitine from softgel capsules is about 100%.

### Combinations of the Ingredients

It has been surprisingly discovered that combinations of oxidative fat metabolizers, neurotransmitters, and algin or algin equivalents when administered to an animal act synergistically to maintain metabolism. In another embodiment, dietary supplements containing L-carnitine, GABA, kelp and MCT interact in a synergistic way to maintain proper metabolism and in certain instances enhance metabolism. The compositions comprising components of the invention surprisingly enhance vitality, reduce blood fat and help metabolize fat-rich or fried foods.

The invention encompasses methods of maintaining: healthy HDL levels, while decreasing unhealthy LDL levels; healthy triglyceride levels; fat metabolism; healthy weight; memory and attention span; mood and mental stability; energy production with less risk of hypoglycemia; heart muscle function and heartbeat regularity; resilience; and sperm health, motility and function.

The compositions of the invention can perform various useful physiological functions including enhancing vitality, reducing blood fats, and helping metabolize fat-rich foods or fried foods.

### Uses of the Compositions of the Invention

In accordance with the invention, the compositions of the invention is administered to an animal, for example, a mammal, for example, a human, for increasing healthy HDL levels, while decreasing unhealthy LDL levels; increasing healthy triglyceride levels; maintaining fat metabolism; maintaining healthy weight; maintaining memory and attention span; maintaining mood and mental stability; maintaining energy production with less risk of hypoglycemia; maintaining heart muscle function and heartbeat regularity; maintaining resilience; and maintaining sperm health, motility and function.

In other embodiments, the compositions of the invention are administered to a mammal, such as a human, as a preventative measure against such disorders.

In a another embodiment, the compositions of the invention are administered as a preventative measure to a mammal, such as a human, having a genetic predisposition to cardiovascular disease, a dyslipidemia, a dyslipoproteinemia, a disorder of glucose metabolism, metabolic syndrome (i.e., Syndrome X).

In another embodiment, the compositions of the invention are administered as a preventative measure to a human having a non-genetic predisposition to cardiovascular disease, a dyslipidemia, a dyslipoproteinemia, a disorder of glucose metabolism, or metabolic syndrome (i.e., Syndrome X). Examples of such non-genetic predispositions include, but are not limited to, cardiac bypass surgery and percutaneous transluminal coronary angioplasty, which often lead to restenosis, an accelerated form of atherosclerosis; diabetes in women, which often leads to polycystic ovarian disease; and cardiovascular disease, which often leads to impotence. Accordingly, the compositions of the invention may be used for the prevention a disorder and concurrently treating another.

The compositions of the invention enhance healthy weight when combined with diet and exercise.

In one embodiment, the compositions of the invention transport fuel into cells and waste products out of cells. The compositions of the invention protect heart, brain, liver, and kidney from toxic chemicals.

In another embodiment the compositions of the invention support cardiovascular health by enhancing fat burning. The compositions of the invention enhance enzyme functions that metabolize sugars, starches, and other carbohydrates, thereby allowing the heart to pump more strongly and beat more regularly.

In another embodiment, the compositions of the invention remove toxic fatty acids from the mitochondria thereby enhancing cell energy production.

In another embodiment, the compositions of the invention promote quicker post-workout recovery.

In another embodiment, the compositions of the invention increase glutathione production thereby aiding in cell detoxification.

### Administration and Compositions

Due to the activity of the compositions of the invention, the compositions are advantageously useful as dietary supplements. As described above, the compositions of the invention are useful for regulating cell metabolism and maintaining healthy physiology.

The invention provides methods of regulating disorders by administration to a patient of an effective amount of a composition of the invention. The patient is a mammal, including, but not limited, to an animal for example a mammal, such as a human.

In one embodiment, the present compositions are administered orally. Various delivery systems are known, e.g., encapsulation in liposomes, microparticles, microcapsules, capsules, etc., and can be used to administer a compound of the invention. The mode of administration is left to the discretion of the practitioner, and will depend in-part upon the site of the medical condition. In most instances, administration will result in the release of the compounds of the invention into the bloodstream.

In another embodiment, the compounds of the invention can be delivered in a vesicle, for example a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.). Each of these documents is herein incorporated by reference in its entirety for all purposes.

In yet another embodiment, the compounds of the invention can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of the compounds of the invention, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used. Each of these documents is herein incorporated by reference in its entirety for all purposes.

As used herein the term “ingredient of the composition of the present invention” comprises any one of an oxidative fat metabolizer, e.g. L-carnitine, a near a transmitter, e.g. GABA, and algin or algin equivalent, e.g. kelp extract, a MCT, e.g. from coconut oil, and optionally, one or more excipients/fillers, e.g. phosphatidylcholine, inositol, ethanolamine, turmeric, beeswax, gelatin, glycerine from e.g. palm fruit, glycerol ethyl ester, water, and combinations thereof which may include any or all of the ingredients that comprise the compositions of the invention.

The present compositions will contain an effective amount of the ingredients of the composition of the invention, optionally more than one ingredient. The ingredients, for example, may be present in purified form, together with a suitable amount of a pharmaceutically acceptable excipient or filler as described herein, so as to provide the form for proper administration to the patient.

In another embodiment, the ingredients of the compositions of the present invention are formulated in accordance with routine procedures as a nutraceutical composition adapted for oral administration to human beings. The compositions of the invention may be administered orally. Compositions for oral delivery may be in the form of pills, tablets, lozenges, aqueous or oily suspensions, granules, powders, emulsions, capsules, syrups, or elixirs, for example. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, when in tablet or pill form, the compositions may be coated to delay disintegration and absorption in the gastrointestinal tract, thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can also include standard additives such as mannitol, lactose, starch, magnesium stearate, sodium saccharine, cellulose, magnesium carbonate, etc. Such vehicles can be of pharmaceutical grade.

The amount of a compound of the invention that will be effective in the regulating a disorder or condition disclosed herein will depend on the nature of the disorder or condition, and can be determined by standard techniques. The precise dose to be employed in the compositions will also depend on the route of administration, and the seriousness of the disease or disorder, and should be decided according to the judgment of the practitioner and each patient's circumstances. However, suitable dosage ranges for oral administration are generally about 1000 mg to about 30 g, about 2000 mg to about 25 g, about 2000 mg to about 20 g, about 2000 mg to about 16 g, about 2000 mg to about 14 g, about 2000 mg to about 11 g, about 2000 mg to about 8 g, about 2000 mg to about 5.5 g, inclusive of all ranges and subranges therebetween.

The dosage amounts described below refer to the amounts of each compound administered; that is, if more than one compound of the invention is administered, the dosages correspond to each amount of the compounds of the invention administered. Oral compositions contain 10% to 95% active ingredient by weight.

The compositions of the invention are administered to regulate disorders. Thus, the compositions of the invention may be administered by any number of routes, including, but not limited to, topical, dermal, subdermal, transdermal, parenteral, oral, rectal, or slow release formulation. The compositions are usually employed in the form of nutraceutical compositions optionally along with a suitable carrier.

Due to the activity of the compositions of the invention, they are useful in administration to animals and humans. The compositions of the invention may be administered by any convenient route, for example, orally, topically, by intravenous infusion or bolus injection, by absorption through epithelial or mucocutaneous linings (e.g., oral mucosa, rectal and intestinal mucosa, etc.) and may be administered together with another biologically active agent.

In one embodiment, the compositions of the invention are administered orally. Administration can be systemic or local. Various delivery systems are known, e.g., encapsulation in liposomes, microparticles, microcapsules, capsules, etc., and can be used to administer a composition of the invention. In certain embodiments, more than one composition of the invention is administered to a patient. Methods of administration include, but are not limited to, intradermal, intramuscular, intraperitoneal, intravenous, subcutaneous, intranasal, epidural, oral, sublingual, intranasal, intracerebral, intravaginal, transdermal, rectally, by inhalation, or topically, to for example the ears, nose, eyes, scalp, or skin. The mode of administration is left to the discretion of the practitioner, and will depend in-part upon the site of the condition. In most instances, administration will result in the release of the composition of the invention for maximum uptake by a cell.

In specific embodiments, it may be desirable to administer one or more compositions of the invention locally to the area in need of treatment. This may be achieved, for example, and not by way of limitation, by topical application (e.g., as a cream); by local infusion during surgery (e.g., in conjunction with a wound dressing after surgery); by injection; by means of a catheter; by means of a suppository; or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

In another embodiment, the composition is prepared in a form suitable for administration directly or indirectly to surface areas of the body for direct application to affected areas. This formulation includes, but is not limited to, anti-drying agents (e.g., pantethine), penetration enhancers (e.g., dimethyl isosorbide), accelerants (e.g., isopropylmyristate) or other common additives that are known in the industry and used for topical applications (e.g., glycerin, propylene glycol, polyethylene glycols, ethyl alcohol, liposomes, lipids, oils, creams, or emollients). In addition, the delivery vehicles of the invention may include compounds that have a beneficial effect on skin pores, such as retinoic acid (i.e., Retin-A), which removes sebum plugs from pores; antioxidants (e.g., butylated hydroxyanisole); or chelating preservatives (e.g., disodium EDTA).

Addition of various concentrations of the enhancer glycerin has been shown to enhance the penetration of cyclosporin (Nakashima et al., 1996). The use of terpene-based penetration enhancers with aqueous propylene glycol have also shown the capacity to enhance topical delivery rates of 5-fluorouricil (Yamane et al., 1995). 5-fluorouricil, 5-FU, is a model compound for examining the characteristics of hydrophilic compounds in skin permeation studies. Thus, the addition of terpenes in polylene glycol (up to 80%) were able to enhance the flux rate into skin.

Dimethyl isosorbide (DMI) is another penetration enhancer that has shown promise for pharmaceutical formulations. DMI is a water-miscible liquid with a relatively low viscosity (Zia et al., 1991). DMI undergoes complexation with water and polylene glycol but not polyethylene glycol. It is the ability for DMI to complex with water that provides the vehicle with the capacity to enhance the penetration of various steroids. Maximum effects were seen at a DMI:water ratio of 1:2. Evidence in the literature suggests that the effect of pH on DMI is an important consideration when using DMI in various formulations (Brisaert et al., 1996).

Pulmonary administration can also be employed, (e.g., by use of an inhaler or nebulizer), and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

Pulmonary administration can also be employed, (e.g., by use of an inhaler or nebulizer), and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the compositions of the invention can be delivered in a vesicle, in for example a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compositions of the invention can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target area to be treated, (e.g., the liver), thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

The present compositions can take the form of solutions, suspensions, emulsion, tablets, pills, pellets, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin.

The amount of a composition of the invention that will be effective in the treatment of a disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. Effective doses may be extrapolated from dose-response curves derived from in vitro or animal model test systems. Such animal models and systems are well known in the art.

In the case of parenteral administration the compositions of the invention may be encapsulated in a liposome “envelope” that is coupled to an antibody directed against human prostate-specific proteins so as to provide target cell selectivity. The specific nature of the formulation is determined by the desired route of administration, e.g., topical, parenteral, oral, rectal, surgical implantation or by other means of local (intraprostatic) delivery. The dosage is determined for the route of administration.

Compositions for rectal administration are prepared with any of the usual pharmaceutical excipients, including for example, binders, lubricants and disintegrating agents. The composition may also include cell penetration enhancers, such as aliphatic sulfoxides. In another embodiment, the composition of the present invention is in the form of a suppository.

Kits of the Invention

The invention also provides pharmaceutical packs or kits comprising one or more containers filled with one or more compounds of the invention. Optionally associated with such container(s) can be a notice, which notice reflects use or sale for human administration. In a certain embodiment, the kit contains more than one compound of the invention. For example, one container of the kit can contain one or more of the individual components of the composition of the present invention, and one or more additional containers can contain the remaining components of the composition of the present invention.

In one embodiment, the ingredients of the composition are separated, optionally in premeasured amounts, within a single container. Instructions detailing the correct assembly and use of the composition may be included in the kit. A kit with separated ingredients would allow the user to combine the composition according to any of the embodiments, thereby allowing flexibility in what is utilized. Users with allergies or possible sensitive reactions would be able to remove any optional ingredients, while still being able to use the invention effectively.

In another embodiment, the ingredients of the composition are partially combined, optionally in premeasured amounts, within a single container. Instructions may be included in the kit to detail the correct assembly and use of the composition in some of its embodiments. While retaining some selective ingredient advantages, users of this type of kit would still be allowed some freedom to tailor the composition according to some embodiments.

The above embodiments may further comprise optionally including instructions related to use. These instructions may include directions on how to prepare the above embodied compositions for a variety of different uses and preferences for combining the ingredients of the composition to optimize a particular use. Directions may also detail the method of administering the composition according the above disclosed methods. Additionally, directions may disclose the sequential or combined administration methods for all or any of the combinations of ingredients of the composition, when the ingredients are provided in separated or partially pre-combined form.

In a further embodiment, some or all of the ingredients of the invention are precombined in a form ready for use. Separate kits may be created to contain one or several different embodiments of the invention. Such kits allow the user to quickly administer an chosen embodiment of the composition while maintaining choices between the quick administration of several different embodiments as well.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

### EXAMPLE

An example of a composition of the present invention is shown in Table 1, below:

| | Amount (per | |
|---|---|---|
| Ingredient | softgel capsule) | |
| L-carnitine (from 862 mg of L-carnitine | 500 | mg |
| fumarate) | | |
| Gamma Aminobutyric Acid (GABA) | 200 | mg |
| Alginate (kelp extract) | 110 | mg |
| Phosphatidylcholine, inositol, | 400 | mg |
| ethanolamine | | |
| Glycerol (100% vegetable from raw palm | 180 | mg |
| fruit) | | |
| Medium Chain Triglycerides (MCT from | 800 | mg |
| pure, raw coconut oil) | | |
| Yellow beeswax | 2 | mg |
| Kosher gelatin | 500 | mg |
| Water | 20 | mg |
| Turmeric powder | 12 | mg |
| Glycerol ethyl ester | 12 | mg |

Various references have been cited herein, each of which is incorporated herein by reference in its entirety.

What is claimed is:

1. A composition comprising at least:
one or more oxidative fat metabolizers;
one or more neurotransmitters;
one or more algins or algin equivalents; and
one or more medium chain triglycerides.

2. A composition according to claim 1, wherein the oxidative fat metabolizer is L-carnitine.

3. A composition according to claim 2, wherein the composition L-carnitine comprises about 10-20% of L-carnitine.

4. A composition according to claim 1, wherein the neurotransmitter is GABA.

5. A composition according to claim 4, wherein the composition comprises about 5-25% of GABA.

6. A composition according to claim 1, wherein the algin or algin equivalent is extracted from kelp.

7. A composition according to claim 6, wherein the composition comprises about 2-5% of the algin or algin equivalent.

8. A composition according to claim 1, wherein the medium chain triglyceride is coconut oil.

9. A composition according to claim 8, wherein the composition comprises 25-45% of the MCT.

10. A composition according to claim 1, further comprising at least one of the following:
phosphatidylcholine, inositol, ethanolamine; and combinations thereof.

11. A composition according to claim 10, wherein the composition comprises about 2-15% of an additional ingredient selected from the group consisting of phosphatidylcholine, inositol, ethanolamine; and combinations thereof.

12. A composition according to claim 1, further comprising:
turmeric.

13. A composition according to claim 12, wherein the composition comprises about 0.1-1% of turmeric.

14. A composition according to claim 1, further comprising at least one of the following:
beeswax, glycerol, gelatin, glycerol ethyl ester, water and combinations thereof.

15. A composition according to claim 14, wherein the composition comprises:
about 0.05-0.5% beeswax;
about 5-15% glycerol;
about 15-20% gelatin;
about 0.1-1% glycerol ethyl ester; and
about 0.5-2% water.

16. A composition according to claim 1, further comprising an pharmaceutically acceptable excipient or filler.

17. A composition comprising:
one or more oxidative fat metabolizers, wherein the oxidative fat metabolizer is L-carnitine and is present in the composition in an amount of from about 16% to about 17%;
one or more neurotransmitters, wherein the neurotransmitter is GABA and is present in the composition in an amount of from about 6% to about 7%;
one or more algins or algin equivalents wherein the algin or algin equivalent is extracted from kelp and are present in the composition in an amount of from about 3% to about 4%;
one or more medium chain triglycerides, wherein the medium chain triglyceride is coconut oil and is present in the composition in an amount of from about 26% to about 28%;
phosphatidylcholine, inositol and ethanolamine in a combined amount of from about 13% to about 14%;
turmeric in an amount of from about 0.3% to about 0.5%;
beeswax in an amount of from about 0.06% to about 0.07%;
gelatin in an amount of from about 16% to about 17%;
glycerol from palm fruit in an amount of from about 13% to about 14%; glycerol ethyl ester in an amount of from about 0.3% to about 0.5%; and water in an amount of from about 0.6% to about 0.7%.

18. A composition according to claim 17, further comprising one or more pharmaceutically acceptable excipients or fillers.

19. A composition according to claim 18, in softgel capsule form.

20. A kit for regulating a condition in a mammal comprising:
a container comprising at least the following components:
one or more oxidative fat metabolizers;
one or more neurotransmitters;
one or more algins or algin equivalents;
one or more medium chain triglycerides; and
instructions for use,
wherein each of the components is pre-measured into a respective unit of use amount.

21. A kit according to claim 20, further comprising at least one of the following components selected from the group consisting of:
phosphatidylcholine, inositol, ethanolamine, turmeric, beeswax, glycerol, gelatin, glycerol ethyl ester, water and combinations thereof,
wherein each of the additional components are pre-measured into a respective unit of use amount.

22. A method of regulating or altering metabolism in a subject comprising administering an effective amount of the composition of claim 1 to a subject.

23. The method of claim 22 wherein the metabolism related disorder is selected from the group consisting of obesity; hyperlipidemia; hypertriglyceridemia; diabetes; atherosclerotic cardiovascular diseases; weight gain; lipid atheromas; hypercholesterolemia; fat embolism; fatty deposits; plaque adhering to arterial walls; Syndrome X; Metabolic Syndrome; defective glucose metabolism; insulin resistance; elevated blood pressure; hypertension; blood lipid imbalance; dyslipidemia; coronary heart disease; cardiomyopathy; cardiac arrhythmia; congestive heart failure; hypoglycemia; low sperm motility; memory; attention span; senility; learning disabilities; brain-blood flow disorders; Alzheimer's disease; motor control; vision disorders; cortical functions; anxiety related disorders; digestion related disorders; circulation related disorders; toxic metabolite related disorders; arthritis; rheumatoid arthritis; osteoarthritis; degenerative joint disorders; muscle weakness; fatigue; malabsorption syndromes; pancreatic disorders; hepatic disorders; gastrointestinal disorders; disorders of the lymph system; seizure disorders; panic attacks; agoraphobia; dementia; mental disturbances; depression; panic; obsessive-compulsive disorder; hepatic and cardiac conditions induced by medication, alcohol, pollution, virus, and toxins; other diseases that are affected by glucose metabolism and/or elevated glucose levels and other metabolic disorders.
